(19) United States
(12) Patent Application Publication
(10) Pub. No.: US 2004/0258649 A1
(43) Pub. Date: Dec. 23, 2004

(54) Aqueous formulations comprising combinations of anionic and cationic surfactants for generating a yeild point

(75) Inventors: Jorg Peggau, Essen (DE); Jurgen Meyer, Essen (DE)

Correspondence Address: SCULLY SCOTT MURPHY & PRESSER, PC, 400 GARDEN CITY PLAZA, GARDEN CITY, NY, 11530

(73) Assignee: GOLDSCHMIDT AG, ESSEN (DE)

(21) Appl. No.: 10/819,751

(22) Filed: Apr. 7, 2004

(30) Foreign Application Priority Data
Apr. 7, 2003 (DE) 103 16 001.9

(51) Int. Cl. A61K 7/075; A61K 7/08

(52) U.S. Cl. 424/70.21; 424/70.22

(57) ABSTRACT

An aqueous formulations including, a) at least one anionic surfactant; and b) at least one cationic surfactant, wherein a) to b) are present in said formulation in a ratio of a) to b) effective for generating a yield point is provided. The aqueous formulations may further include at least one co-surfactant, auxiliaries and/or additives.

### FIELD OF THE INVENTION

The present invention relates to aqueous cleaning formulations that include a coherent liquid phase and a disperse solid, liquid or gaseous phase in which particular combinations of anionic and nonionic or cationic co-surfactants are used to generate a yield point.

### BACKGROUND OF THE INVENTION

Formulations containing yield points belong to known prior art and are widely used in cosmetic and pharmaceutical applications, for the cleaning of hard surfaces, and in the coatings industry.

Systems of this type are used primarily for stably and homogeneously distributing dispersed constituents in a coherent liquid phase.

Dispersed constituents which may be co-used are pigments, dyes, polymers, optionally encapsulated active ingredients or enzymes, abrasives, plant extracts, or gaseous or liquid components for producing sensory effects.

The known formulations are usually based on macromolecules which, in adequate concentrations, form a three-dimensional network and thus form a yield point in the systems to be stabilized. The macromolecules used are usually hydroxyethylcellulose, carmellose sodium (cellulose gum), and carbomers (polyacrylates). More recently, a combination of chitosan and EDTA has also been proposed.

Less customary are inorganic additives based on phyllosilicates (bentonites, clays), highly disperse silicon dioxide, which in principle can likewise form yield points in aqueous formulations, but in some cases form unacceptable gels in some formulations, or which produce colored opaque gels due to their intrinsic color and particle size.

The disadvantage of using customary polymer additives is their sensitivity toward electrolytes, in particular toward the anionic surfactants, which are obligatory in cleaning applications.

A disadvantage in anionic-surfactant-rich systems is that at high concentrations of polymeric thickeners, considerable increases in viscosity result, which considerably impair handlability (pumping, pouring, metering). Despite the high viscosity, it is also not always possible to generate yield points in electrolyte- or surfactant-rich systems.

High viscosities in these formulations are sometimes desired and even necessary in order to delay separation phenomena in disperse systems. However, on their own, they are unable to bring about permanent stabilization.

There is therefore a need for permanently stabilized systems which avoid the disadvantages of the prior art, have an adequate yield point, are of low viscosity under application conditions and thus permit or facilitate handlability, and have tolerance toward increased electrolyte concentrations.

### SUMMARY OF THE INVENTION

This object is achieved in the present invention by producing aqueous formulations comprising a combination of anionic and cationic surfactants, and optionally nonionic or amphoteric co-surfactants.

In broad terms, the present invention provides aqueous formulations comprising

a) at least one anionic surfactant;

b) at least one cationic surfactant in a ratio of a) to b) effective for generating a yield point; and, optionally

c) nonionic and/or amphoteric cosurfactants and/or betaines and, optionally

d) further auxiliaries and additives.

The ratio of a) to b) is largely variable and is dependent on the nature of the surfactants used and on the extent of the desired yield point.

According to the present invention, ratios of a) and b) from 4:1 to 1:4, preferably from 1.8:1 to 1:1.8, are employed. It is necessary in the present invention for the yield point of the formulation is sufficient to hold dispersed particles in a stable manner in the coherent aqueous phase.

In contrast to the solution proposals based on macromolecules, it has surprisingly been found that the combination according to the present invention of anionic and cationic surfactants is by itself adequate for preparing formulations with a yield point which is of relevance in practice and which are able to stabilize dispersed constituents permanently in the coherent phase without the co-use of additional thickeners.

In particular, even at relatively high temperatures from 40° to 60° C., these systems exhibit stable yield points (in contrast to most of the macromolecular thickeners). In this connection, these formulations are notable for the fact that they become flowable even under the action of small forces and thus have relatively low viscosities.

A further advantage of the present invention is the surprisingly high electrolyte compatibility of these formulations toward the electrolyte concentrations, as usually arise in cleaning formulations (mostly >1% salt).

As aqueous cleaning formulations, these surfactant systems may be used in the field of anionic-surfactant-containing formulations customary for household and industrial cleaners. The amounts of cationic surfactants and optionally co-surfactants required to generate a yield point, and also, where required, further auxiliaries and additives, are added to these formulations.

### BRIEF DESCRIPTION OF THE DRAWINGS

FIG. 1 is a plot of the measured rheological properties, i.e., frequency-dependent oscillating measurement at 25° C., of inventive formulation A5.

FIG. 2 is a plot of the measured rheological properties, i.e., frequency-dependent oscillating measurement at 25° C., of inventive formulation A6.

FIG. 3 is a plot of deformation vs. shear stress for formulations A5 and A6 at shear ramps from 1 to 200 Pa in 200 s at 25° C.

FIG. 4 is a viscosity curve of selected formulations from Table 2.

### DETAILED DESCRIPTION OF THE INVENTION

The yield point in the composition according to the present invention means that solid, liquid or gaseous substances can be distributed in surfactant systems in a stable manner irrespective of the difference in density. Various groups of substances can, as a result, be used in customary surfactant systems and be stabilized.

Liquid/solid insoluble active ingredients:

These include skin protectants that bring about protective, care or physiological properties, such as, e.g., creatine, phytosphingosines, ceramides, plant extracts or vitamins.

Insoluble solids or crystalline substances:

These include cleaning-boosting substances, such as, abrasives, e.g., marble flour, PUR flour, metal oxides, such as, titanium dioxide and aluminum oxides, aluminum silicates. Crystalline substances are also used in order to achieve optical effects, such as, e.g., ethylene glycol distearate as a pacifier or pearlizing agent or as visible solid particles which, being colored, give the product a particular appearance, and also microencapsulated active ingredients, such as, e.g., percarbonate or chloroisocyanurate.

Gaseous substances:

This is understood essentially as meaning air bubbles intentionally introduced during the preparation process, which give the product a particular brilliant appearance. This effect is used, for example, with hair gels, in addition also gaseous fragrances, which are released when such products are handled.

For all of these substances, an unstable distribution would have a negative effect in terms of performance and/or feel. Examples thereof are, e.g., pearlescence-containing dishwashing compositions or shower gels in which the separation can even be detected visually. Examples of performance-reduced products are a scouring milk or a polish which no longer comprise abrasive agents in use.

The composition of said products, in particular those with a high electrolyte content and anionic surfactant content, may make the use of customary thickeners more difficult or render it ineffective. The use of more than 1% of anionic surfactants, which is customary in particular in cleaning compositions, prevents the generation of a yield point necessary for the stabilization in the case of most polymeric thickeners. Even if the viscosity of the products is increased, this does not lead to storable products. In practice, this is compounded also by the marked temperature dependency of customary thickeners.

However, using the combinations of anionic and cationic surfactants according to the present invention it is possible, even in these problematic cases, to ensure flowable formulations with an adequate yield point.

Some of these quaternary compounds surprisingly exhibit good solubility in the combinations according to the present invention and, even in low concentrations, have a thickening effect (cf. A14 and A15). In these low concentrations, the quaternary compounds do not exhibit a yield point; this is only the case at increased concentration (cf A8). The yield point is achieved after passing through the phases from clear via cloudy to clear again. This property is exhibited primarily by quaternary compounds with an alkyl chain smaller than C₁₀ and here primarily the compounds C₆ to C₁₀, such as, e.g., bis(octylcarboxyethyl)-hydroxyethyl-methylammonium methosulfates and bis(octylcarboxyiso-propyl ester) dimethylammonium methosulfates. In combination with anionic surfactants, these compounds exhibit an excellent thickening behavior. Upon adding amounts of from 3 to 10% of cationic surfactant to the anionic active ingredient content, these solutions are clear and thickening.

Example: With an anionic active ingredient content of 10%, added amounts of from 0.3 to 1% have a thickening effect. Above 1%, these solutions are cloudy and clarify again only at about 6%.

The present invention therefore further provides for the use of aqueous formulations comprising

a) at least one anionic surfactant; and

b) at least one cationic surfactant

in lower concentrations than for achieving the yield point as thickeners.

The formulations according to the present invention comprise surfactants in a total amount of customarily 0.5 to 60% by weight, preferably 10 to 45% by weight and most preferably 12 to 40% by weight. Standard commercial household cleaners usually comprise about 8 to 30% by weight of surfactants.

Anionic Surfactants a) which can be Co-Used according to the Present Invention include:

The alkyl ether sulfates, alkyl- and/or arylsulfonates and/or alkyl sulfates and the other anionic surfactants are usually used as the alkali metal, alkaline earth metal and/or mono-, di- or trialkanolammonium salt and/or, however, also in the form of their corresponding acid to be neutralized in situ with the corresponding alkali metal hydroxide, alkaline earth metal hydroxide and/or mono-, di- or trialkanolamine. Preferred alkali metals here are potassium and, in particular, sodium, preferred alkaline earth metals are calcium and, in particular, magnesium, and preferred alkanolamines are mono-, di- or triethanolamine. Particular preference is given to the sodium salts.

Alkyl Ether Sulfates:

Alkyl ether sulfates (fatty alcohol ether sulfates, INCI Alkyl Ether Sulfates) are products of sulfation reactions on alkoxylated alcohols. In this connection, the person skilled in the art generally understands alkoxylated alcohols as meaning the reaction products of alkylene oxide, preferably ethylene oxide, with alcohols, for the purposes of the present invention preferably with longer-chain alcohols, i.e., with aliphatic straight-chain or mono- or polybranched, acyclic or cyclic, saturated or mono- or polyunsaturated, preferably straight-chain, acyclic, saturated, alcohols having 6 to 22, preferably 8 to 18, in particular 10 to 16 and particularly preferably 12 to 14, carbon atoms. As a rule, n moles of ethylene oxide and one mole of alcohol produces, depending on the reaction conditions, a complex mixture of addition products of varying degrees of ethoxylation (n=0 to 30, preferably 0.3 to 20, in particular 0.3 to 10, particularly preferably 0.3 to 5). A further embodiment of the alkoxylation consists in the use of mixtures of alkylene oxides, preferably the mixture of ethylene oxide and propylene oxide. For the purposes of the present invention, very particular preference is given to fatty alcohols with low degrees of ethoxylation having 0.3 to 4 ethylene oxide units (EO), in particular 0.3 to 2 EO, for example 0.5 EO, 1.0 EO, 1.3 EO and/or 2.0 EO, such as Na C₁₂₋₁₄-fatty alcohol+0.5 EO sulfate, Na C₁₂₋₁₄-fatty alcohol+1.3 EO sulfate, Na C₁₂₋₁₄-fatty alcohol+2.8 EO sulfate and/or Mg C₁₁₋₁₄-fatty alcohol +1.0 EO sulfate.

The composition according to the present invention can comprise one or more alkyl ether sulfates in an amount of usually 1 to 50% by weight, preferably 3 to 40% by weight, in particular more than 6 to 30% by weight, particularly preferably 8 to 20% by weight, most preferably 10 to 16% by weight.

Alkyl- and/or Arylsulfonates:

The alkyl sulfonates (INCI Sulfonic Acids) usually have an aliphatic straight-chain or a mono- or polybranched, acyclic or cyclic, saturated or mono- or polyunsaturated, preferably branched, acyclic, saturated, alkyl radical having 6 to 22, preferably 9 to 20, in particular 11 to 18 and particularly preferably 13 to 17, carbon atoms. Suitable alkylsulfonates are accordingly the saturated alkanesulfonates, the unsaturated olefinsulfonates and the ether sulfonates—which are derived formally from the alkoxylated alcohols which also form the basis of the alkyl ether sulfates—for which a distinction is made between terminal ether sulfonates (n-ether sulfonates) with sulfonate function bonded to the polyether chain, and internal ether sulfonates (i-ether sulfonates) with sulfonate function linked to the alkyl radical.

According to the present invention, preference is given to the alkanesulfonates, in particular alkanesulfonates with a branched, preferably secondary, alkyl radical, for example the secondary alkanesulfonate secondary Na C₁₃₋₁₇-alkanesulfonate (INCI Sodium C₁₄₋₁₇-Alkyl Sec-Sulfonate).

Preferably used arylsulfonates are alkylbenzene-sulfonates, where the alkyl radicals are branched and unbranched chains having C₁₋₂₀, preferably C₂₋₁₈, particularly preferably C₆₋₁₆ and most preferably C₈₋₁₂. Particularly preferred examples here are linear alkylbenzenesulfonates (LAS) and/or cumenesulfonates.

Alkyl Sulfates:

In the present invention, it is also possible to use alkyl sulfates, such as, e.g., fatty alcohol sulfates. Suitable alkyl sulfates are sulfates of saturated and unsaturated fatty alcohols having C₆₋₂₂, preferably C₁₀₋₁₈ and particularly preferably C₁₂₋₁₆. Particularly suitable alkyl sulfates are those with native C cut C₁₂₋₁₄₋₁₆ and/or petrochemical C cut C₁₂₋₁₃/C₁₄₋₁₅ in the range from 0 to 15%, preferably 0 to 10%, particularly preferably 0 to 8%.

The composition according to the present invention can comprise one or more of these compounds in an amount of from usually 0.1 to less than 50% by weight, preferably 2 to 10% by weight.

Cationic surfactants b) which can be co-used according to the invention include: The composition according to the invention can additionally comprise one or more cationic surfactants (INCI Quaternary Ammonium Compounds), usually in an amount of from 0.5 to 50% by weight, preferably 8 to 15% by weight.

Particularly preferred cationic surfactants are quaternary ammonium compounds (QAC) at times antimicrobially effective ammonium compounds (QAC; INCI Quaternary Ammonium Compounds) according to the general formula:

(R^{I})(R^{II})(R^{III})(R^{IV})N⁺X⁻

in which

R^{I} to R^{IV} are identical or different C₁₋₂₂-alkyl radicals, C₇₋₂₀-aralkyl radicals or heterocyclic radicals, where two or in the case of an aromatic incorporation, such as pyridine, even three radicals, together with the nitrogen atom, form the heterocycle, e.g., a pyridinium or imidazolinium compound, and

X− is halide ions, sulfate ions, hydroxide ions or similar anions. For optimum antimicrobial effect, at least one of the radicals preferably has a chain length of from 8 to 18, in particular 12 to 16, carbon atoms.

QACs can be prepared by reacting tertiary amines with alkylating agents, such as, e.g., methyl chloride, benzyl chloride, dimethyl sulfate, dodecyl bromide, but also ethylene oxide. The alkylation of tertiary amines with one long alkyl radical and two methyl groups proceeds particularly readily, and the quaternization of tertiary amines with two long radicals and one methyl group can also be carried out using methyl chloride under mild conditions. Amines which have three long alkyl radicals or hydroxy-substituted alkyl radicals are not very reactive and are preferably quaternized using dimethyl sulfate.

Preferred QACs are imidazolinium quats, esters of alkanolamines and fatty acids, so-called ester quats, in particular the di- and triesters of triethanolamine (TEA), methyldiethanolamine (MDEA), the di-and triesters of triisopropanolamine, methyldiisopropanol-amine or alkoxylated derivatives thereof, in particular the ethoxylates and propoxylates.

According to the present invention, preference is given to TEA ester quats based on fatty acids having 6 to 22, in particular 8 to 18, carbon atoms, which, for reasons of good environmental compatibility, are especially preferred in household cleaning compositions as QACs.

These cationic surfactants are used in amounts of from 0.1% by weight to 30% by weight, preferably 0.5 to 20% by weight, in particular <15% by weight.

Optionally Co-Used Nonionic and/or Amphoteric Cosurfactants c) Include:

The composition present according to the invention may comprises one or more amphoteric surfactants in an amount of from customarily 0.1 to 20% by weight, preferably 1 to 15% by weight, in particular 2 to 12% by weight, particularly preferably 3 to 10% by weight, most preferably 4 to 8% by weight.

Nonionic surfactants for the purposes of the present invention are fatty alcohol glycol ethers and fatty acid alkanolamides and fatty acid polyglycol ethers. Important classes of nonionic surfactants according to the present invention are also the amine oxides and the sugar surfactants, in particular the alkyl polyglucosides. Particular preference is also given to using sorbitan esters, such as, e.g., sorbitan monoalkylates with C₈₋₁₈ alkyl radicals.

Fatty alcohol polyglycol ethers are, according to the present invention, understood as meaning branched or unbranched, saturated or unsaturated C₁₀₋₂₂-alcohols alkoxylated with ethylene oxide (EO) and/or propylene oxide (PO) with a degree of alkoxylation up to 30, preferably ethoxylated C₁₀₋₁₈-fatty alcohols with a degree of ethoxylation of less than 30, preferably with a degree of ethoxylation of from 1 to 20, in particular from 1 to 12, particularly preferably from 1 to 8, most preferably from 2 to 5, for example C₁₋₁₄-fatty alcohol ethoxylates with 2, 3 or 4 EO or a mixture of the C₁₂₋₁₄-fatty alcohol ethoxylates with 3 and 4 EO in the weight ratio of 1 to 1 or isotridecyl alcohol ethoxylate with 5, 8 or 12 EO, fatty acid polyethylene glycol ethers consisting of C₈₋₁₈-alkyl and alkenyl radicals with a degree of ethoxylation of >20 EO.

Amine oxides which are suitable according to the present invention include alkylamine oxides, in particular alkyldimethylamine oxides, alkylamidoamine oxides and alkoxyalkylamine oxides.

Preferred amine oxides are those according to the general formula

R⁶R⁷R⁸N⁺—O−

R⁶—C(O)—NH—[(CH₂)—N(R⁹)—]_{z}—N⁺(R⁷)(R⁸)—O⁻

in which

R⁶ is a saturated or unsaturated C₆₋₂₂-alkyl radical, preferably C₈₋₁₈-alkyl radical, in particular a saturated C₁₀₋₁₆-alkyl radical, for example a saturated C₁₂₋₁₄-alkyl radical, which is bonded to the nitrogen atom N in the alkylamidoamine oxides via a carbonylamidoalkylene group —CO—NH—(CH₂)_{z}—, and in the alkoxyalkylamine oxides via an oxyalkylene group —O—(CH₂)_{z}—, where

z is in each case a number from 1 to 10, preferably 2 to 5, in particular 3, and

R⁹ is H and/or R⁷, R⁸ and

R⁷, R⁸, independently of one another, is a C₁₋₄alkyl radical, optionally hydroxy-substituted, such as e.g. a hydroxyethyl radical, in particular a methyl radical.

A preferred amine oxide is, for example, Cocamidopropyl-amine Oxide (cocoamidopropylamine oxide).

Amphoteric Surfactants (Amphoteric surfactants, Zwitterionic Surfactants):

These include betaines, alkylamidoalkylamines, alkyl-substituted amino acids, acylated amino acids or biosurfactants, of which preference is given to the betaines for the purposes of this invention.

Betaines:

Suitable betaines are the alkylbetaines, the alkylamido-betaines, the imidazoliniumbetaines, the sulfobetaines (INCI Sultaines), and also the phosphobetaines and preferably satisfy formula (A)

R¹—[CO—X—(CH₂)ₙ]ₓ—N⁺(R²)(R³)—(CH₂)ₘ—[CH (OH)—CH₂)_{y}—Y⁻ (A)

in which

R¹ is a saturated or unsaturated C₈₋₂₂-alkyl radical, preferably a saturated C₁₀₋₁₆-alkyl radical,

X is NH, NR⁴ with the C₁₋₄-alkyl radical R⁴, O or S,

n is a number from 1 to 10, preferably 2 to 5, in particular 3,

x is 0 or 1, preferably 1,

R², R³, independently of one another, is a C₁₋₄-alkyl radical, optionally hydroxy-substituted, such as e.g. a hydroxyethyl radical, but in particular a methyl radical,

m is a number from 1 to 4, in particular 1, 2 or 3,

y is 0 or 1 and

Y⁻is COO, SO₃, OPO(OR⁵)O or P(O)(OR⁵)O, where R⁵ is a hydrogen atom H or a C₁₋₄-alkyl radical.

These alkyl- and alkylamidobetaines with a carboxylate group (Y⁻=COO⁻) are also called carbobetaines. Preferred amphoteric surfactants are the alkylbetaines of formula (A), the alkylamidobetaines of formula (B), the sulfobetaines of formula (C), the amidosulfobetaines of formula (D) and the amidosulfobetaines of formula (E),

(Aa) R¹—N⁺(CH₃)₂—CH₂COO⁻

(Ab) R¹—CO—NH—(CH₂)₃—N⁺(CH₃)₂—CH₂COO^{—}

(Ac) R¹—N⁺(CH₃)₂—CH₂CH(OH)CH₂SO₃—

(Ad) R¹—CO—NH—(CH2)₃—N⁺(CH₃)₂—CH₂CH(OH)CH₂SO₃—

(Ae) (CH₃)3—Si—O—[SiR(CH₃)—O]m—[Si(CH₃)2—O]n—Si(CH₃)₃ (TEGOPREN® 6950)

in which R¹ has the same meaning as in formula (A).

Particularly preferred amphoteric surfactants are the carbobetaines, in particular the carbobetaines of formula (Aa), most preferably the alkylamidobetaines of formula (Ab).

Examples of suitable betaines and sulfobetaines are the compounds below, named in accordance with INCI: Almondamidopropyl-Betaine, Apricotamidopropyl-Betaine, Avocadamidopropyl-Betaine, Babassuamidopropyl-Betaine, Behenamidopropyl-Betaine, Behenyl-Betaine, Betaine, Canolamidopropyl-Betaine, Capryl/Capramidopropyl-Betaine, Carnitine, Cetyl-Betaine, Cocamidoethyl-Betaine, Coca-midopropyl-Betaine, Cocamidopropyl-Hydroxysultaine, Coca-Betaine, Coco-Hydroxysultaine, Coco/Oleamidopropyl-Be-taine, Coco-Sultaine, Decyl-Betaine, Dihydroxyethyl-Oleyl-Glycinate, Dihydroxyethyl-Soy-Glycinate, Dihydroxy-ethyl-Stearyl-Glycinate, Dihydroxyethyl-Tallow-Glycinate, Dimethicone-Propyl-PG-Betaine, Erucamidopropyl-Hydroxy-sultaine, Hydrogenated-Tallow-Betaine, Isostearamido-propyl-Betaine, Lauramidopropyl-Betaine, Lauryl-Betaine, Lauryl-Hydroxysultaine, Lauryl-Sultaine, Milkamidopropyl-Betaine, Minkamidopropyl-Betaine, Myristamidopropyl-Betaine, Myristyl-Betaine, Oleamidopropyl-Betaine, Oleamidopropyl-Hydroxysultaine, Oleyl-Betaine, Olivamido-propyl-Betaine, Palmamidopropyl-Betaine, Palmitamido-propyl-Betaine, Palmitoyl-Camitine, Palm-Kemelamido-propyl-Betaine, Polytetrafluoroethylene-Acetoxypropyl-Betaine, Ricinoleamidopropyl-Betaine, Sesamidopropyl-Betaine, Soyamidopropyl-Betaine, Stearamidopropyl-Betaine, Stearyl-Betaine, Tallowamidopropyl-Betaine, Tallowamidopropyl-Hydroxysultaine, Tallow-Betaine, Ta-llow-Dihydroxyethyl-Betaine, Undecylenamidopropyl-Betaine and Wheat-Germamidopropyl-Betaine. A preferred betaine is, for example, Cocamidopropyl-Betaine (cocoamidopropyl-betaine), silicone betaine, such as, for example, Dimethicone-Propyl-PG-Betaine, such as TEGOPREN® 6950.

Alkylamidoalkylamines:

The alkylamidoalkylamines (INCI Alkylamido Alkylamines) are amphoteric surfactants of formula (B)

R⁹—CO—NR¹⁰—(CH₂)ᵢN(R¹¹)—(CH₂CH₂O)ⱼ—(CH₂)ₖ—[CH(OH)]₁—CH₂—Z—OM [symbol] (B)

in which

R⁹ is a saturated or unsaturated C₆₋₂₂-alkyl radical, preferably C₈₋₁₈-alkyl radical, in particular a saturated C₁₀₋₁₆-alkyl radical, for example a saturated C₁₂₋₁₄-alkyl radical,

R¹⁰ is a hydrogen atom H or a C₁₋₄-alkyl radical, preferably H,

R¹¹ is a hydrogen atom H or CH₂COOM (for M see below),

i is a number from 1 to 10, preferably 2 to 5, in particular 2 or 3,

j is a number from 1 to 4, preferably 1 or 2, in particular 1,

k is a number from 0 to 4, preferably 0 or 1,

l is 0 or 1, where k is 1 when 1 is 1,

Z is CO, SO₂, OPO(OR¹²) or P(O)(OR¹²), where R¹² is a C₁₋₄-alkyl radical or M (see below), and

M is a hydrogen, an alkali metal, an alkaline earth metal or a protonated alkanolamine, e.g., protonated mono-, di- or triethanolamine.

Preferred representatives satisfy formulas (Ba) to (Bd),

(Ba) R⁹—CO—NH—(CH₂)₂—N(R¹¹)—CH₂CH₂O—CH₂—COOM

(Bb) R⁹—CO—NH—(CH₂)₂—N(R¹¹)—CH₂CH₂O—CH₂CH₂—COOM

(Bc) R⁹—CO—NH—(CH₂)₂—N(R¹¹)—CH₂CH₂O—CH₂(OH)CH₂—SO₃M

(Bd) R⁹—CO—NH—(CH₂)₂N(R¹¹)—CH₂CH₂O—CH₂CH₂(OH)CH₂—OPO₃HM

in which R¹¹ and M have the same meanings as in formula (B).

Exemplary alkylamidoalkylamines are the compounds below, named in accordance with INCI:

Cocoamphodipropionic Acid, Cocobetainamido Amphopro-pionate, DEA-Cocoamphodipropionate, Disodium Capro-amphodiacetate, Disodium Caproamphodipropionate, Disodium Capryloamphodiacetate, Disodium Caprylo-amphodi-propionate, Disodium Coco-amphocarboxy-ethylhydroxy-propylsulfonate, Disodium Cocoamphodiacetate, Disodium Cocoamphodipropionate, Disodium Isostearo-amphodiacetate, Disodium Isostearoamphodipropionate, Disodium Laureth-5 Carboxyamphodiacetate, Disodium Lauro-amphodiacetate, Disodium Lauroamphodipropionate, Disodium Oleoampho-dipropionate, Disodium PPG-2-lsodeceth-7 Carboxy-amphodiacetate, Disodium Stearoamphodiacetate, Disodium Tallowamphodiacetate, Disodium Wheat Germamphodiacetate, Lauroamphodipropionic Acid, Quaternium-85, Sodium Caproamphoacetate, Sodium Caproamphohydroxypropyl-sulfonate, Sodium Caproamphopropionate, Sodium Caprylo-amphoacetate, Sodium Capryloamphohydroxy-propylsulfonate, Sodium Capryloamphopropionate, Sodium Cocoam-phodi-acetate, Sodium Cocoamphohydroxy-propylsulfonate, Sodium Cocoamphopropionate, Sodium Cornamphopropionate, Sodium Isostearoamphodiacetate, Sodium Isostearoamphopropionate, Sodium Lauroamphodi-acetate, Sodium Lauroamphohydroxy-propylsulfonate, Sodium Lauroampho PG-Acetate Phosphate, Sodium Lauro-amphopropionate, Sodium Myristoamphodi-acetate, Sodium Oleoamphodiacetate, Sodium Oleoamphohy-droxypropyl-sulfonate, Sodium Oleoamphopropionate, Sodium Ricinoleoamphodiacetate, Sodium stearoamphodiacetate, Sodium Stearoamphohydroxypropylsulfonate, Sodium Stearo-amphopropionate, Sodium Tallamphopropionate, Sodium Tallowamphodiacetate, Sodium Undecylenoamphodiacetate, Sodium Undecylenoamphopropionate, Sodium Wheat Germamphodiacetate and Trisodium Lauroampho PG-Acetate Chloride Phosphate.

Alkyl-Substituted Amino Acids:

Alkyl-substituted amino acids (INCI Alkyl-Substituted Amino Acids) preferred according to the present invention are monoalkyl-substituted amino acids according to formula (C),

R¹³—NH—CH(R¹⁴)—(CH₂)ᵤ—COOM′ (C)

in which

R¹³ is a saturated or unsaturated C₆₋₂₂-alkyl radical, preferably C₈₋₁₈-alkyl radical, in particular a saturated C₁₀₋₁₆-alkyl radical, for example a saturated C₁₂₋₁₄alkyl radical,

R¹⁴ is a hydrogen atom H or a C₁₋₄-alkyl radical, preferably H,

u is a number from 0 to 4, preferably 0 or 1, in particular 1, and

M′ is a hydrogen, an alkali metal, an alkaline earth metal or a protonated alkanolamine, e.g. protonated mono-, di- or triethanolamine,

and alkyl-substituted imino acids according to formula (D)

R¹⁵—N—[(CH₂)ᵥ—COOM″]₂ (D)

in which

R¹⁵ is a saturated or unsaturated C₆₋₂₂-alkyl radical, preferably C₈₋₁₈alkyl radical, in particular a saturated C₁₀₋₁₆-alkyl radical, for example a saturated C₁₂₋₁₄alkyl radical,

v is a number from 1 to 5, preferably 2 or 3, in particular 2, and

M″ is a hydrogen, an alkali metal, an alkaline earth metal or a protonated alkanolamine, e.g. protonated mono-, di- or triethanolamine, where M″ in the two carboxyl groups may have the same meaning or two different meanings, e.g. hydrogen and sodium or both sodium,

and mono- or dialkyl-substituted natural amino acids according to formula (E),

R¹⁶—N(R¹⁷)—CH(R¹⁸)—COOM′″ (E)

in which

R¹⁶ is a saturated or unsaturated C₆₋₂₂-alkyl radical, preferably C₈₋₁₈-alkyl radical, in particular a saturated C₁₀₋₁₆ -alkyl radical, for example a saturated C₁₂₋₁₄-alkyl radical,

R¹⁷ is a hydrogen atom or a C₁₋₄alkyl radical, optionally hydroxy- or amine-substituted, e.g. a methyl, ethyl, hydroxyethyl or aminopropyl radical,

R¹⁸ is the radical of one of the 20 natural α-amino acids H₂NCH(R¹⁸)COOH, and

M′″ is a hydrogen, an alkali metal, an alkaline earth metal or a protonated alkanolamine, e.g. protonated mono-, di- or triethanolamine.

Particularly preferred alkyl-substituted amino acids are the aminopropionates according to formula (Ea),

R¹³—NH—CH₂CH₂COOM′ (Ea)

in which

R¹³ and M′ have the same meanings as in formula (E).

Exemplary Alkyl-Substituted Amino Acids are the Compounds Below, Named in Accordance with INCI:

Aminopropyl Laurylglutamine, Cocaminobutyric Acid, Cocaminopropionic Acid, DEA-Lauraminopropionate, Disodium Cocaminopropyl Iminodiacetate, Disodium Di-carboxyethyl Cocopropylenediamine, Disodium Lauriminodipropionate, Disodium Steariminodipropionate, Disodium Tallowimino-dipropionate, Lauraminopropionic Acid, Lauryl Amino-propylglycine, Lauryl Diethylenediaminoglycine, Myrist-aminopropionic Acid, Sodium C₁₂₋₁₅-Alkoxypropyl-Iminodi-propionate, Sodium Cocaminopropionate, Sodium Laur-aminopropionate, Sodium Lauriminodipropionate, Sodium Lauroyl Methylaminopropionate, TEA-Lauraminopropionate and TEA-Myristaminopropionate.

Acylated Amino Acids:

Acylated amino acids are amino acids, in particular the 20 natural α-amino acids, which carry, on the amino nitrogen atom, the acyl radical R¹⁹CO of a saturated or unsaturated fatty acid R¹⁹COOH, where R¹⁹ is a saturated or unsaturated C₆₋₂₂-alkyl radical, preferably C₈₋₁₈-alkyl radical, in particular a saturated C₁₀₋₁₆-alkyl radical, for example a saturated C₁₂₋₁₄-alkyl radical. The acylated amino acids can also be used in the form of the alkali metal salt, alkaline earth metal salt or alkanolammonium salt, e.g., mono-, di- or triethanolammonium salt. Exemplary acylated amino acids are the acyl derivatives listed according to INCI under Amino Acids, e.g., Sodium Cocoyl Glutamate, Lauroyl Glutamic Acid, Capryloyl Glycine or Myristoyl Methylalanine.

The nonionic and amphoteric cosurfactants may be used either individually or else in mixtures with one another. In a particular embodiment, the composition according to the present invention comprises one or more amphoteric surfactants in an amount of more than 8% by weight. In yet a further particular embodiment, the composition according to the present invention comprises one or more amphoteric surfactants in an amount of less than 5% by weight.

The coherent outer phase of the formulations according to the present invention consists primarily of water and optionally of co-used auxiliaries, such as, for example, mono- or polyfunctional alcohols, which optionally comprise one or more ether groups, such as, for example, the known polyoxyalkylene alcohols.

Suitable polyalkylene alcohols are, for example, saturated or unsaturated, preferably saturated, branched or unbranched C₁₋₂₀-hydrocarbons, preferably C₂₋₁₀-hydrocarbons, with at least one hydroxyl group and optionally one or more ether functions C—O—C, i.e., oxygen atoms interrupting the carbon atom chain.

Preferred polyoxyalkylene alcohols are the C₂₋₆-alkylene glycols and poly-C₂₋₃-alkylene glycol ethers optionally etherified on one side with a C₁₋₆-alkanol and having, on average, 1 to 9 identical or different, preferably identical, alkylene glycol groups per molecule, and also the C₁₋₆-alcohols, preferably ethanol, propanol or butanol, in particular n-butanol.

Particularly preferred polyoxyalkylene alcohols are the poly-C₂₋₃-alkylene glycol ethers etherified on one side with a C₁₋₆alkanol and having, on average, 1 to 9, preferably 2 to 3, ethylene or propylene glycol groups, for example PPG-2 methyl ether (dipropylene glycol monomethyl ether). Particular preference is also given to using dipropylene glycol n-butyl ether.

These additives are usually used in amounts of from 0 to 20%.

According to the present invention, particular preference is accordingly given to mixtures of anionic, cationic, and the specified cosurfactants.

particular preference is given to the formulation specified in table 1.

TABLE 1

| | Composition in % by wt. | | | | | | | | |
|---|---|---|---|---|---|---|---|---|---|
| | A1 | A2 | A3 | A4 | A5 | A6 | A7 | V1 | V2 |
| Na C₁₂₋₁₄-fatty alcohol + 1.3 | 10.7 | 5.8 | 5.8 | 8 | 8.2 | 8.2 | 8.2 | 8.2 | 8.2 |
| EO sulfate | | | | | | | | | |
| Sec. Na C₁₂₋₁₆-alkanesulfonate | 5.3 | 2.9 | 2.9 | 4 | 4.1 | 4.1 | 4.1 | 4.1 | 4.1 |
| Bis(oleylcarboxyethyl)- | 16.7 | — | 9.0 | 12.5 | 12.1 | 12.1 | — | | |
| hydroxyethyl-methylammonium | | | | | | | | | |
| methosulfate* | | | | | | | | | |
| Bis(cocoylcarboxy- | — | 9.0 | — | — | — | — | 10.0 | 1.5 | — |
| ethyl)hydroxyethyl- | | | | | | | | | |
| methylammonium methosulfate | | | | | | | | | |
| Cocamidoalkylbetaine | 5.3 | 2.9 | 2.9 | 4 | 4.1 | 4.1 | 4.1 | 4.1 | 4.1 |
| C₈₋₁₈-Fatty acid ethoxylate | — | — | 3.0 | — | 0.1 | — | — | — | — |
| Sorbitan monopalmitate | — | — | — | — | — | 0.1 | — | — | — |
| Dipropylene glycol n-butyl ether | 5.0 | 5.0 | 3.0 | 7.5 | 1.0 | 4.0 | 7.0 | — | — |
| Dipropylene glycol monomethyl | 5.0 | — | — | — | — | — | — | — | — |
| ether | | | | | | | | | |
| Xanthan gum | — | — | — | — | — | — | — | — | 2.5 |
| Ethanol | 4 | 4 | 4 | 4 | 4 | 4 | 4 | 4 | 4 |
| Citric acid monohydrate | 0.2 | 0.2 | 0.2 | 0.2 | 0.2 | 0.2 | 0.2 | 0.2 | 0.2 |
| Water, perfume, dye, | ad | ad | ad | ad | ad | ad | ad | ad | ad |
| preservative | 100 | 100 | 100 | 100 | 100 | 100 | 100 | 100 | 100 |
| Yield point 1-200 Pa in 200 s | 3 Pa | 1 Pa | 2 Pa | 2 Pa | 3 Pa | 5 Pa | 5 Pa | — | — |
| [25° C.] | | | | | | | | | |
| Viscosity at a shear rate of 10/s | 3.5 | 2.1 | 1.9 | 3.8 | 3.3 | 3.5 | 4.1 | 3.5 | 1.3 |
| [Pa · s] | | | | | | | | | |

*prepared from mixed fatty acid

TABLE 2

| | Composition in % by wt. | | | | | | | | |
|---|---|---|---|---|---|---|---|---|---|
| | A8 | A9 | A10 | A11 | A12 | A13 | A14 | A15 | V3 |
| Na C₁₂₋₁₄-fatty alcohol + 2.8 | 8.0 | 8.0 | 6.8 | 8.0 | 8.0 | 8.0 | 8.0 | 8.0 | 8.0 |
| EO sulfate | | | | | | | | | |
| Sec. Na C₁₂₋₁₆-alkanesulfonate | 4.0 | 4.0 | 3.2 | 4.0 | 4.0 | 4.0 | 4.0 | 4.0 | 4.0 |
| Cocamidoalkylbetaine | 4.0 | 4.0 | 0.9 | 4.0 | 4.0 | 4.0 | 4.0 | 4.0 | 4.1 |
| Bis(oleylcarboxyethyl)- | | 8.0 | 27.7 | | | | | | |
| hydroxyethylmethyl-ammonium | | | | | | | | | |
| methosulfate** | | | | | | | | | |
| Bis(octylcarboxyethyl)- | 7.0 | | | | | | 1.5 | 0.8 | |
| hydroxyethylmethyl-ammonium | | | | | | | | | |
| methosulfate | | | | | | | | | |
| Bis(oleylcarboxyiso-propyl | | | | 8.8 | | | | | |
| ester)dimethyl-ammonium | | | | | | | | | |
| methosulfate | | | | | | | | | |
| 1-Methyl-2-noroleyl-3-oleic | | | | | 9.0 | | | | |
| acid-amidoethyl-imidazolinium | | | | | | | | | |
| methosulfate | | | | | | | | | |
| Dicocodimethylammonium | | | | | | 4.8 | | | |
| chloride | | | | | | | | | |
| Dipropylene glycol n-butyl | 11.0 | 7.0 | 1.6 | 7.0 | 7.0 | 7.0 | 0.4 | 0.2 | — |
| ether | | | | | | | | | |
| Citric acid monohydrate | 0.2 | 0.2 | 0.2 | 0.2 | 0.2 | 0.2 | 0.2 | 0.2 | 0.2 |
| Water, perfume, dye, | ad | ad | ad | ad | ad | ad | ad | ad | ad |
| preservative | 100 | 100 | 100 | 100 | 100 | 100 | 100 | 100 | 100 |
| Yield point 1-200 Pa in 200 s | 0.8 | 1.0 | — | 4.5 | n.B. | n.B. | — | — | — |
| [25° C.] | | | | | | | | | |
| Viscosity at a shear rate of 10/s | 0.2 | 2.1 | 0.2 | 4.8 | 2.1 | 3.5 | 3.5 | 1.0 | 0.08 |
| [Pa · s] | | | | | | | | | |

**prepared from pure oleic acid

Using rheological measurements it is possible to show that all of the samples according to the present invention have yield points. Samples V1, V2 and V3 are comparison products which, although they are viscous, do not exhibit yield points.

Measurement of the Yield Point:

The steady state of the formulations can be characterized with the help of oscillating rheological measurements. For this purpose, an oscillating shear stress amplitude is placed on the sample which is sufficiently small not to disturb the steady-state structure of the samples (the samples were measured at 0.5 Pa). The applied shear stress amplitude brings about a phase-shifted deformation movement the sample. From the ratios of the applied shear stress amplitude and the measured deformation amplitude, it is possible to determine the corresponding shear moduli. These shear moduli are complex parameters (contain an elastic portion and a viscous portion). From the measured phase shift of deformation and shear stress, it is then possible to determine these portions. In this connection, the storage modulus G′ describes the elastic portions of a sample, and the loss modulus G″ describes the viscous portions of a sample.

Measurement at different frequencies allows an insight into how a sample behaves in the case of a short-term acting force (high frequencies), and as to how a sample behaves under long-term stress (low frequencies).

A classic yield point is evident in these measurements from the elastic portion being clearly above the viscous portion over the entire frequency domain (G′>G″). At the same time, a yield point which has long-term stability is evident from the fact that G′ scarcely decreases even at low frequencies.

The complex viscosity decreases in the log-log plot over the entire frequency domain and increases for low frequencies toward infinity. This is another typical indication of systems with yield points.

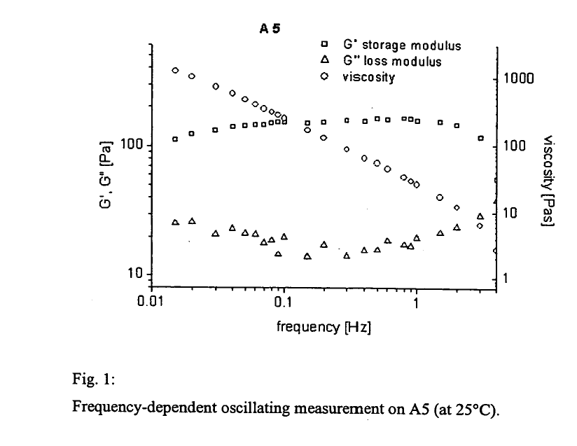

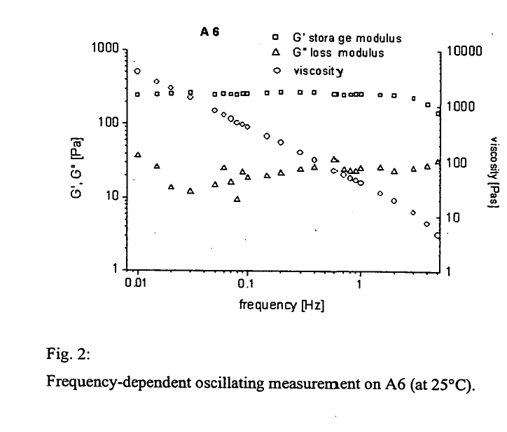

The formulations according to the present invention are notable for the presence of such yield points (see, e.g., FIG. 1 and FIG. 2).

As well as this meaningful qualitative indication of yield points, which primarily takes into consideration the character of such parameters with respect to time, it is also possible to determine numerical values for such yield points, which have also been listed in Table 1.

However, in this connection, it should be taken into consideration that the numerical value of a yield point determined in this way is always dependent on the nature of the experiment, the nature of the plot and the rate at which the experiment was carried out (time dependency).

To determine the yield points, shear stress ramps from 1 to 200 Pa were applied (rotational measurement), which were passed through in a period of 200 seconds (at 25° C.).

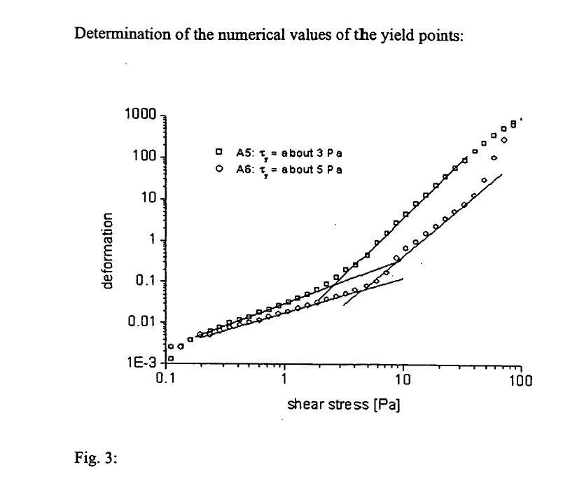

In this connection, the detected deformation of the sample was plotted against the applied shear stress in a log-log diagram (FIG. 3).

Below the yield point (elastic behavior), the deformation of the samples increases linearly (analogously to the deflection of a spring). Above the yield point, the sample starts to flow, which leads to this linear deflection being abandoned (steep increase in the deformation above a certain shear stress τ_{y}; τ_{y}=yield point).

Samples A5 and A6 may be singled out as examples.

Both formulation A5 and formulation A6 exhibit those oscillating rheograms typical of systems with yield points (FIGS. 1 and 2).

Both formulations thus exhibit yield points with long-term stability, as are required for the stabilization according to the invention of disperse phases.

For A5 the yield point is thus about 3 Pa, and for A6 the yield point is about 5 Pa.

It can be readily seen how the higher yield point of A5 correlates with the higher values of G′ for A6 in the oscillating measurements.

The high effectiveness of the dicocodimethylammonium chloride (A13) and of the bis(octylcarboxyethyl)-hydroxyethylmethylammonium methosulfate (A8) at which even a 50% reduced concentration is sufficient to achieve a significant effect, as evident in the summary of the results, was surprising.

Measurement of the Viscosity Curve:

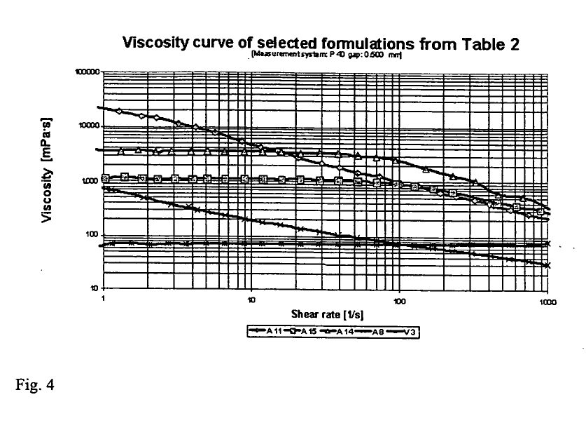

Since this type of thickening system involves non-Newtonian liquids, the rheological measurements additionally included a viscosity curve in order to describe the yield behavior upon shearing. Here, the formulations A8 and A11, which are characterized by yield points, exhibit no viscosity plateau, which was also to be expected with the prior findings. The formulations A14 and A15 exhibit a viscosity plateau, but no yield point. With these added amounts it is possible to thicken anionic/amphoteric surfactant mixtures very effectively, which was very surprising for the product group described. The comparison solution V3 describes Newtonian viscosity behavior. By means of these specified examples, the diverse modifications of the yield behavior of such solutions have been adequately described. FIG. 4 shows the viscosity curve of selected formulations from Table 2. Measurement system p$0 gap:0.500 mm.

While the present invention has been particularly shown and described with respect to illustrative and preferred embodiments thereof, it will be understood by those skilled in the art that the foregoing and other changes in form and detail may be made therein without departing from the spirit and scope of the invention. Thus, the present invention should be limited only by the scope of the appended claims.

1. An aqueous formulation having yield points and comprising
a) at least one anionic surfactant; and
b) at least one cationic surfactant,
wherein a) to b) are present in said formulation in a ratio of a) to b) effective for generating a yield point.

2. The aqueous formulation of claim 1, wherein the ratio of a) and b) is from 4:1 to 1:4.

3. The aqueous formulation of claim 1, wherein the ratio of a) and b) is from 1.8:1 to 1:1.8.

4. The aqueous formulation of claim 1, wherein the at least one anionic surfactant comprises at least one compound from the group of fatty alcohol ether sulfates, secondary alkanesulfonates, or secondary alkenesulfonates.

5. The aqueous formulation of claim 1, wherein the at least one cationic surfactant comprises at least one alkanolamine ester quat.

6. The aqueous formulation of claim 5, wherein the at least one cationic surfactant comprises at least one triethanolamine ester quat.

7. The aqueous formulation of claim 1 further comprising, as component c), at least one of a nonionic surfactant or an amphoteric surfactant.

8. The aqueous formulation of claim 7, wherein component c) comprises at least one of (poly)sorbitan esters, fatty alcohol alkoxylates, fatty acid alkoxylates, betaines, amphoteric surfactants, or amine oxides.

9. The aqueous formulation of claim 1 further comprising, as component d), at least one auxiliary or additive.

10. The aqueous formulation of claim 9, wherein component d) comprises one of short-chain mono- or polyhydric alcohols or alkoxylates.

11. An aqueous formulation having yield points comprising
a) 8 to ≦30% by weight of at least one anionic surfactant;
b) 3 to 15% by weight of at least one cationic surfactant, in a ratio of a) to b) effective for generating a yield point;
c) 0 to 15% by weight of at least one of a nonionic or amphoteric co-surfactant; and
d) 0 to 20% by weight of further auxiliaries and additives
e) ad 100% by weight of water.

12. A cleaning or care agent for use in a preparation for technical, cosmetic, pharmaceutical or surface-active applications comprising an aqueous formulation having yield points, said formulation having a) at least one anionic surfactant; and b) at least one cationic surfactant, wherein a) to b) are present in said formulation in a ratio of a) to b) effective for generating a yield point.
